(19) United States
(12) Patent Application Publication
(10) Pub. No.: US 2019/0021820 A1
(43) Pub. Date: Jan. 24, 2019

(54) OCCLUSAL SPLINT

(71) Applicant: LUCERA INVESTMENTS SAGL, Lugano (CH)

(72) Inventor: Gennaro LUCERA, Viggiù (Varese) (IT)

(21) Appl. No.: 16/067,040

(22) PCT Filed: Aug. 16, 2016
(86) PCT No.: PCT/IB2016/054902

§ 371 Date: Jun. 28, 2018

(30) Foreign Application Priority Data
Jan. 19, 2016 (CH) 00071/2016

(51) Int. Cl. A61C 7/36; A61F 5/56; A61C 7/08; A61C 5/00

(52) U.S. Cl. CPC A61C 7/36; A61F 5/566; A61F 2005/563; A61C 5/007; A61C 7/08

(57) ABSTRACT

A bite guard includes an upper portion intended to be engaged, under conditions of use of the bite, with the upper dental arch of a wearer, and a lower portion intended to be positioned, under at least one condition of use of the bite, on the lower dental arch of the wearer. The lower portion includes a guide portion configured and intended to bring about a predetermined controlled forward sliding of the wearer's lower jaw relative to the wearer's upper jaw by sliding the lower dental arch forward on the guide portion and provide at least a partial rest for the back surfaces of a plurality of teeth of the lower dental arch of the wearer. The guide portion can be movable relative to the upper portion.

### CROSS-REFERENCE TO RELATED APPLICATIONS

National Stage of International Application No. PCT/162016/054902, filed on Aug. 16, 2016, which claims the benefit of, and priority to Swiss Application No. 00071/2016 filed Jan. 19, 2016.

### TECHNICAL FIELD

This disclosure relates to oral appliances. In particular, this disclosure relates to an orthodontic appliance intended to be interposed between the lower and upper dental arches of a wearer.

### DETAILED DESCRIPTION OF ILLUSTRATIVE EMBODIMENTS

In one general aspect, an occlusal splint, also commonly referred to as a “bite guard” is disclosed. The concepts disclosed herein can also have application in a device known as a “bite plane”. A bite guard is an orthodontic appliance intended to be interposed between the lower and upper dental arches of a wearer in order to modify their reciprocal positioning.

There are known bite guards or orthodontic appliances which serve to reduce the damage deriving from incorrect positions of the dental arches, or to reduce the effects deriving from other problems such as bruxism or teeth grinding. In general, incorrect positions of the dental arches cause tensions in the muscles of the face, neck and oral cavity (tongue muscles), which essential processes such as chewing, swallowing and the articulation of sounds depend on. Such muscle tensions are particularly undesirable, as they can compromise the aforesaid processes. Furthermore, the muscles of the face, neck and oral cavity also act on the spinal column and hence the tension thereof causes stresses which may result in an incorrect alignment of the spinal column and thus problems in terms of posture, balance and walking.

With the aim of re-educating the positioning of the dental arches, the known bite guards act on the relative position of the dental arches.

However, the known bite guards do not ensure a high effectiveness in re-educating the positioning of the dental arches or in postural re-education.

Furthermore, the known bite guards do not enable the positioning of the wearer's lower jaw to be controlled and defined in a precise manner.

Furthermore, the known bite guards do not enable the known problems related to muscle tensions, postural imbalances, bruxism, etc. to be sufficiently reduced.

In this context, the disclosed occlusal splints intend to solve one or more of the problems found in the prior art.

In certain embodiments, the disclosed bite guard is capable of correcting incorrect positions of the wearer's lower jaw.

An additional object of the disclosure is a bite guard capable of reducing or eliminating the muscle tensions deriving from incorrect positions of the dental arches of the wearer and the consequent problems.

A further object of the present disclosure is a bite guard that enables the postural re-education of the wearer.

These objects and others besides, which will become more apparent from the present description, are substantially achieved by a bite guard in accordance with what is expressed in one or more of the appended claims and/or of the disclosed aspects, taken on their own or in any combination with one another, and/or in combination with any of the further aspects or features described herein.

In one exemplary aspect, there is provided a bite guard, that includes:
a lower portion intended to be engaged, under conditions of use of the bite guard, with the lower dental arch of a wearer;
an upper portion intended to be positioned, under at least one condition of use of the bite guard, on the upper dental arch of the wearer; and
a containment portion for the wearer's tongue, in particular an arcuate containment portion, the wearer's tongue being positioned beneath the containment portion under conditions of use of the bite guard.

In one embodiment, the containment portion includes one or more through openings intended to place, under conditions of use of the bite guard, the portion of the oral cavity beneath the containment portion in communication with the portion of the oral cavity above the containment portion.

In one embodiment, the upper portion further includes a fixed ridge disposed externally and frontally relative to said abutment ridge, and wherein the abutment ridge is movable and/or slidable relative to the fixed ridge.

In one embodiment, the adjuster is disposed in a front portion of the bite guard.

In one embodiment, the adjuster is disposed in a lateral portion of the bite guard.

In one embodiment, the adjuster includes at least one threaded adjustment element, in particular an endless screw, in the adjustment configuration said at least one adjustment element acting on the abutment ridge to adjust the position thereof.

In one embodiment, the adjuster includes a fastening element that is selectively actuatable so as to enable the movement and fix the position, respectively, of the abutment ridge.

In one embodiment, the fastening element includes a threaded fastening pin.

In one embodiment, the adjuster includes two threaded adjustment elements disposed in proximity to opposite lateral portions of the bite guard.

In one embodiment, said at least one adjustment element is housed at least partially inside the fixed ridge.

In one embodiment, said at least one adjustment element crosses the fixed ridge.

In one embodiment, the adjuster and/or the adjustment elements extend at least partially on the upper portion of the bite guard.

In one embodiment, the adjuster and/or the adjustment elements extend at least partially on a lateral portion of the bite guard.

In one embodiment, the lower portion and/or the upper portion of the bite guard are made of resin or another certified material compatible for use.

In one embodiment, the abutment ridge is made at least partially of metal material.

In one embodiment, the abutment ridge is coated with resin.

In one embodiment, the abutment ridge includes a resin-coated metal skeleton.

In one embodiment, the containment portion includes at least one reinforcement element, in particular said reinforcement element being made of metal material and extending transversely relative to lateral portions of the bite guard.

In one embodiment, said at least one reinforcement element is embedded in the containment portion.

In one embodiment, the lower portion is at least partially of a shape mating that of the lower dental arch of the wearer.

In one embodiment, the lower portion includes a housing seat intended to house, at least partially, the lower dental arch of the wearer.

In one embodiment, the through holes are positioned in a front portion of the bite guard, in particular in a central portion of the bite guard disposed between the upper portion and the lower portion.

In one embodiment, the appendage extends from a central portion of the bite guard disposed between the upper portion and the lower portion.

In one aspect, the through holes and/or the through channels are disposed in proximity to the upper portion of the bite guard and/or are facing the containment portion from above.

In one embodiment, the bite guard includes abutment elements disposed in the lower portion, said abutment elements being configured to be engaged with the lower dental arch of the wearer in order to bring about a correct positioning of the lower portion relative to the lower dental arch.

In one embodiment, the abutment elements are made of metal material for dental use.

In another exemplary aspect, there is provided a bite guard that includes:
an upper portion intended to be positioned, under at least one condition of use of the bite guard, on the upper dental arch of a wearer; and
a lower portion intended to be positioned, under at least one condition of use of the bite guard, on the lower dental arch of the wearer. The bite guard further includes a positioning element for positioning at least one dental arch of the wearer, the positioning element being configured and intended to bring about a predetermined controlled forward sliding of the wearer's lower jaw relative to the wearer's upper jaw by sliding the lower dental arch forward and provide at least a partial rest for a plurality of teeth of said at least one dental arch of the wearer.

The bite guard and/or the positioning element are configured and intended to bring about the forward sliding of the lower jaw relative to the upper jaw in concomitance with the closure, and thus nearing, of the lower jaw relative to the upper jaw. In one embodiment:
the upper portion is intended to be engaged, under conditions of use of the bite guard, with the upper dental arch of a wearer,
the lower portion is intended to be positioned, under at least one condition of use of the bite guard, on the lower dental arch of the wearer, the positioning element including a guide portion disposed on the lower portion, the guide portion being configured and intended to bring about a predetermined controlled forward sliding of the wearer's lower jaw relative to the wearer's upper jaw by sliding the lower dental arch forward on the guide portion and provide at least a partial rest for the back surfaces of a plurality of teeth of the lower dental arch of the wearer.

In one embodiment, the guide portion has a surface that is inclined relative to a front portion of the bite guard, the inclined surface extending away from the front portion of the bite guard.

In one embodiment, the guide portion extends for a width substantially equal to an extent of the lower dental arch of the wearer.

In one embodiment, the lower portion is fixed and/or is made in one piece with the upper portion, in particular when the bite guard is made in a single piece.

In one embodiment, the lower portion is movable relative to an upper portion in order to vary the reciprocal position between the lower portion and upper portion and enable a plurality of operative positions of the bite guard, the guide portion being solidly joined to the lower portion, a movement of the lower portion bringing about the movement of the guide portion in such a way as to modulate the position of the guide portion relative to the upper portion and bring about, according to the position taken on by the guide portion, a specific sliding of the wearer's lower jaw relative to the upper jaw.

In one embodiment, the bite guard further includes a regulator for adjusting the position of the lower portion, said regulator being configured to enable a variation in the reciprocal position between the lower portion and upper portion.

In one embodiment, the regulator is of a threaded type and includes a screw for adjusting the reciprocal position between the lower portion and upper portion. In one embodiment, the bite guard further includes position indicators configured to indicate the reciprocal position between the lower portion and upper portion, the position indicators preferably being disposed in lateral portions of the bite guard.

In one embodiment, the bite guard further includes:
lateral guide portions configured to enable the reciprocal movement between the lower portion and upper portion, the lateral guide portions being disposed on either the lower portion or upper portion,
lateral sliding portions housed inside the lateral guide portions in such a way as to be able to slide relative to the lateral guide portions, the lateral guide portions being disposed on either the lower portion or the upper portion, the sliding of the lateral portions bringing about the relative sliding between the upper portion and guide portion.

In one embodiment, the lateral guide portions and the lateral sliding portions are made of metal material integrated into the material, preferably plastic, that the lower and upper portions of the bite guard are made of, or wherein the lateral guide portions and lateral sliding portions are shaped portions made of the same material, preferably plastic, as the lower and upper portions of the bite guard.

In one embodiment, the guide portion has one or more through holes.

In one embodiment, the bite guard further includes a deformable or soft portion, having less hardness than the lower portion, said deformable portion being applied frontally relative to the lower portion so as make resting the teeth of the lower arch on the lower portion more comfortable.

In one embodiment, the deformable or soft portion is engaged with the lower portion via one or more through holes of the guide portion.

In one embodiment,
the lower portion is intended to be engaged, under conditions of use of the bite guard, with the lower dental arch of a wearer,
the upper portion is intended to be positioned, under at least one condition of use of the bite guard, on the upper dental arch of the wearer,
   the positioning element including an abutment ridge disposed on the upper portion, said abutment ridge being configured and intended to define a limit position with respect to an advancement of, and a rest for the front surfaces of, a plurality of teeth of the upper dental arch of the wearer, so as to bring about a predetermined controlled forward sliding of the wearer's lower jaw relative to the wearer's upper jaw.

The predetermined controlled forward sliding of the wearer's lower jaw relative to the wearer's upper jaw takes place, for example, in concomitance with the closure, and thus nearing, of the lower jaw relative to the upper jaw and/or in a position of closure of the lower jaw relative to the upper jaw.

In one embodiment, the abutment ridge is configured and intended to define a limit position relative to an advancement of, and a rest for the front surfaces of at least the upper incisors of the wearer and/or wherein the abutment ridge is substantially vertically aligned with or set rearwardly and retracted relative to the lower portion, in such a way as to enable, under conditions of use of the bite guard, the positioning of at least said plurality of teeth and/or of the upper incisors in abutment on the abutment ridge and the positioning of the lower dental arch in a position that is substantially vertically aligned with or advanced relative to the upper dental arch, said positioning bringing about the forward sliding of the wearer's lower jaw.

In one embodiment, the abutment ridge is fixed and/or made in one piece with the upper portion.

In one embodiment, the abutment ridge is configured to be movable and/or slidable relative to the lower portion and positionable in a plurality of progressively rearward and retracted positions relative to the lower portion, so as to enable the position of the abutment ridge to be varied selectively in order to vary, under at least one condition of use of the bite guard, the predetermined controlled forward sliding of the wearer's lower jaw relative to the upper jaw.

In one embodiment, the bite guard including at least one adjuster for adjusting the position of the abutment ridge, the adjuster being selectively actuatable so as to move the abutment ridge into a plurality of progressively retracted operative positions relative to the lower portion and/or acting between an adjustment configuration, in which it enables adjustment of the position of the abutment ridge, and a fixed configuration, in which it does not enable adjustment of the position of the abutment ridge.

In one embodiment,
the bite guard includes lateral guide portions configured to enable the sliding of the abutment ridge,
the abutment ridge includes lateral sliding portions housed inside the lateral guide portions in such a way as to be able to slide relative to the lateral guide portions, the sliding of the lateral portions bringing about the sliding of the abutment ridge.

In one embodiment, the abutment ridge is at least partially of a shape mating that of the upper dental arch of the wearer, in particular of a shape mating that of at least the incisors of the upper dental arch.

In one embodiment, the bite guard further includes a containment portion for the wearer's tongue, in particular an arcuate containment portion, the wearer's tongue being positioned, under conditions of use of the bite guard, beneath the containment portion; preferably, the containment portion includes one or more through openings intended to place, under conditions of use of the bite guard, the portion of the oral cavity beneath the containment portion in communication with the portion of the oral cavity above the containment portion.

In one aspect, the bite guard is provided with one or more through holes disposed in a front portion of the bite guard and intended, under conditions of use of the bite guard, to place the oral cavity of the wearer in communication with the outside environment.

In one embodiment, the bite guard includes an appendage disposed in a front portion of the bite guard and intended, under conditions of use of the bite guard, to be positioned between the wearer's lips in a projecting position relative to the oral cavity in order to provide a rest for the lips, wherein the appendage is preferably provided with one or more through channels suitable for placing the oral cavity of the wearer in communication with the outside environment under conditions of use of the bite guard.

By way of non-limiting example, a detailed description of one or more preferred embodiments of the invention will now be set forth below, wherein:

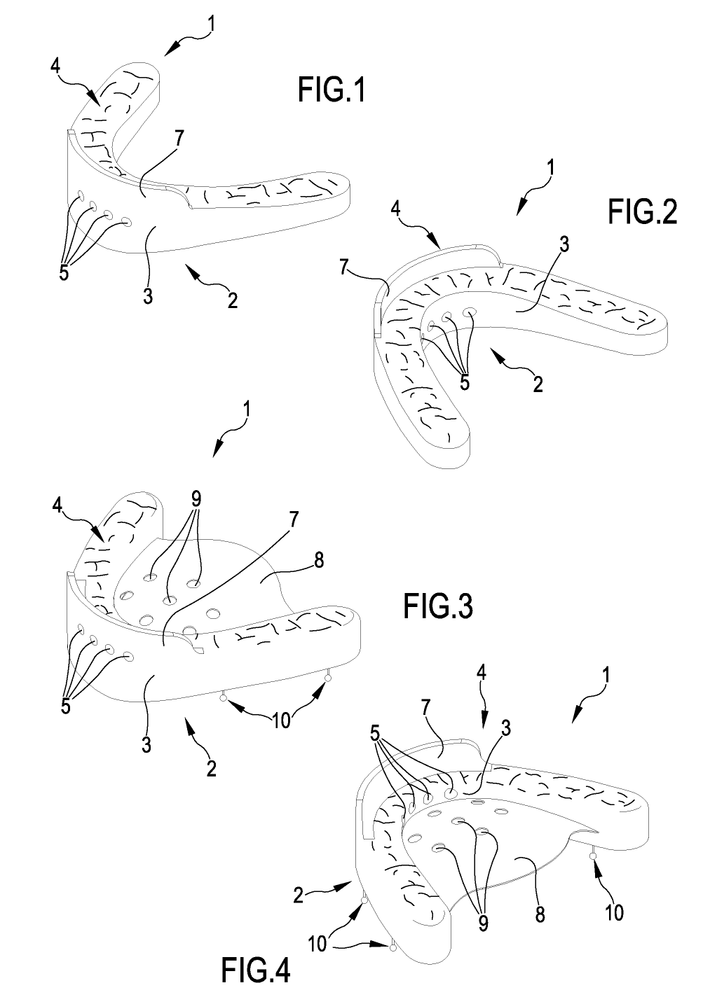

FIGS. 1-2 represent front and rear perspective views, respectively, of a bite guard according to a first embodiment;

FIGS. 3-4 represent front and rear perspective views, respectively, of a bite guard according to a second embodiment;

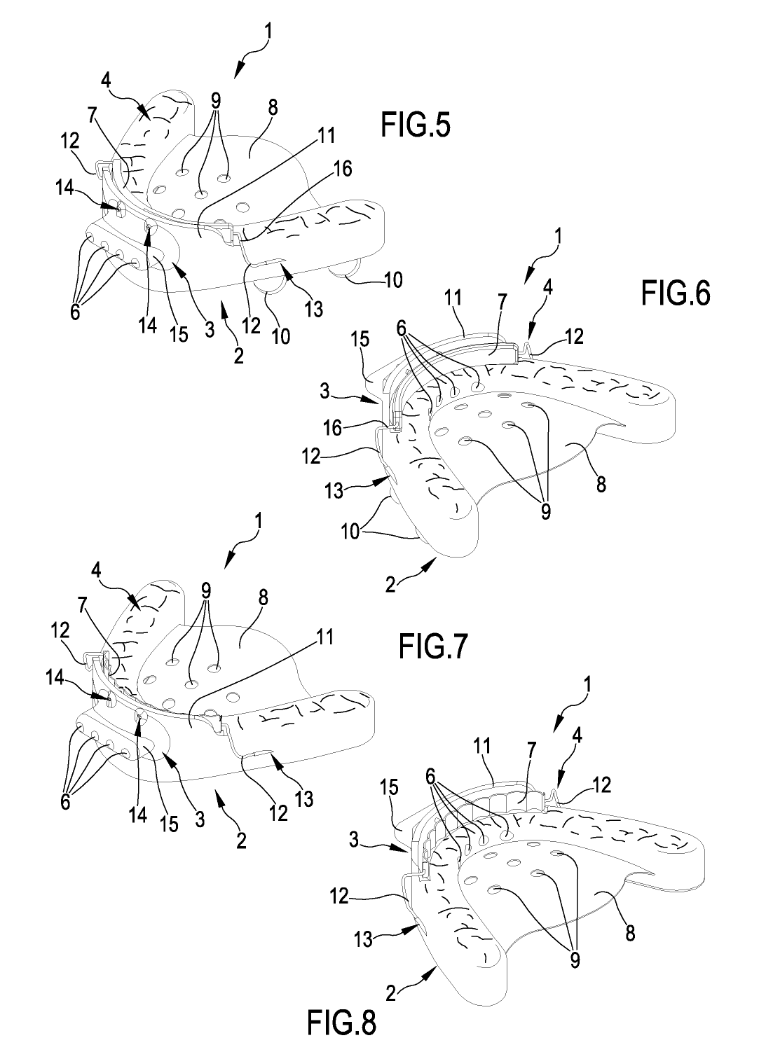

FIGS. 5-6 represent front and rear perspective views, respectively, of a bite guard according to a third embodiment;

FIGS. 7-8 represent front and rear perspective views, respectively, of a bite guard according to a fourth embodiment;

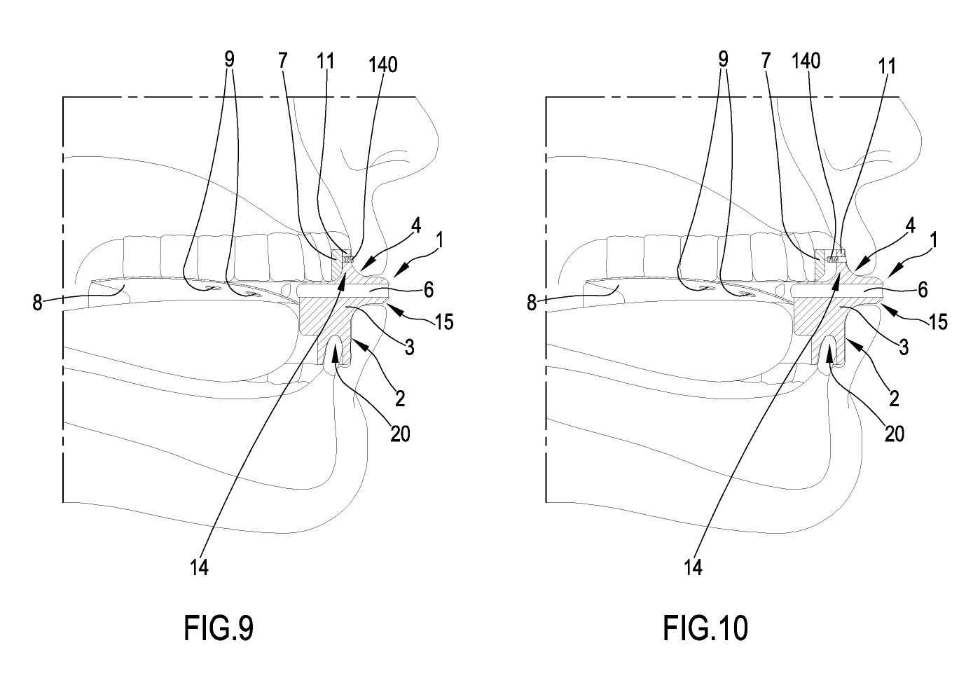

FIGS. 9-10 represent a bite guard according to the fourth embodiment under exemplary conditions of use;

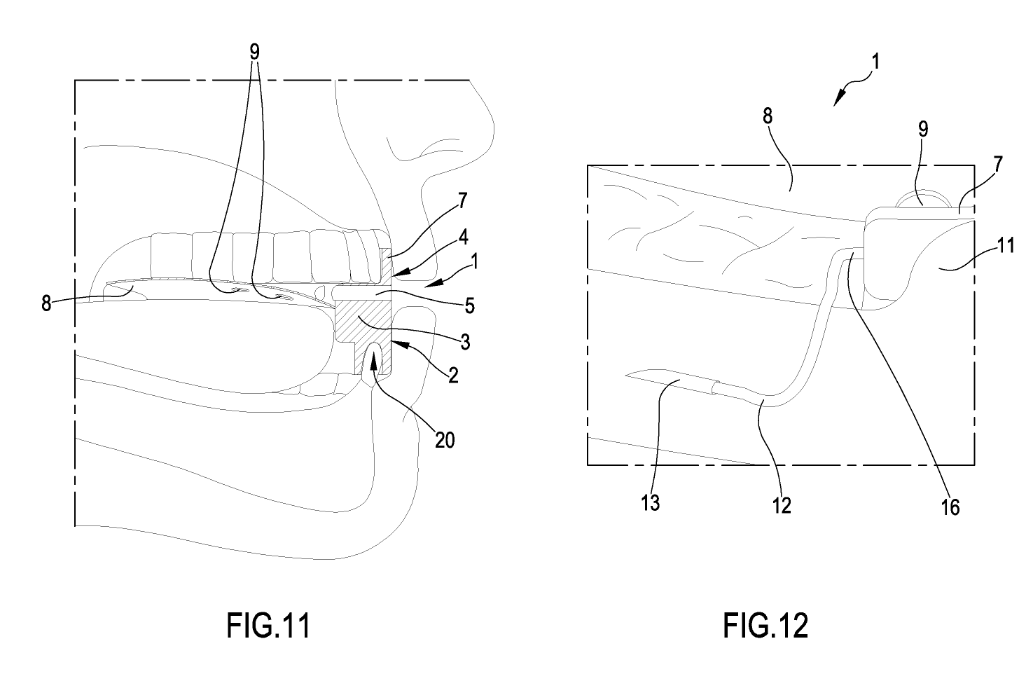

FIG. 11 represents a bite guard according to the second embodiment under exemplary conditions of use;

FIG. 12 represents a detail of the engagement of the abutment ridge with lower portion of the bite guard according to the third embodiment;

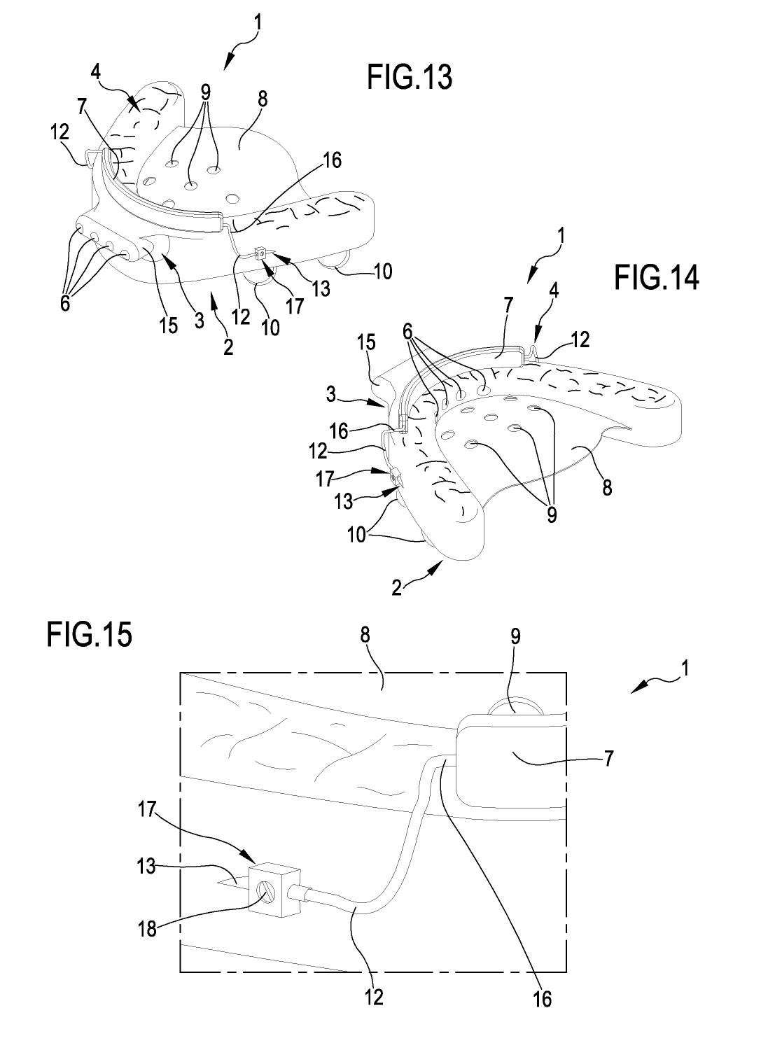

FIGS. 13-14 represent front and rear perspective views, respectively, of a bite guard according to a fifth embodiment;

FIG. 15 represents an exemplary detail of the engagement of the abutment ridge with the lower portion of the bite guard according to the fifth embodiment;

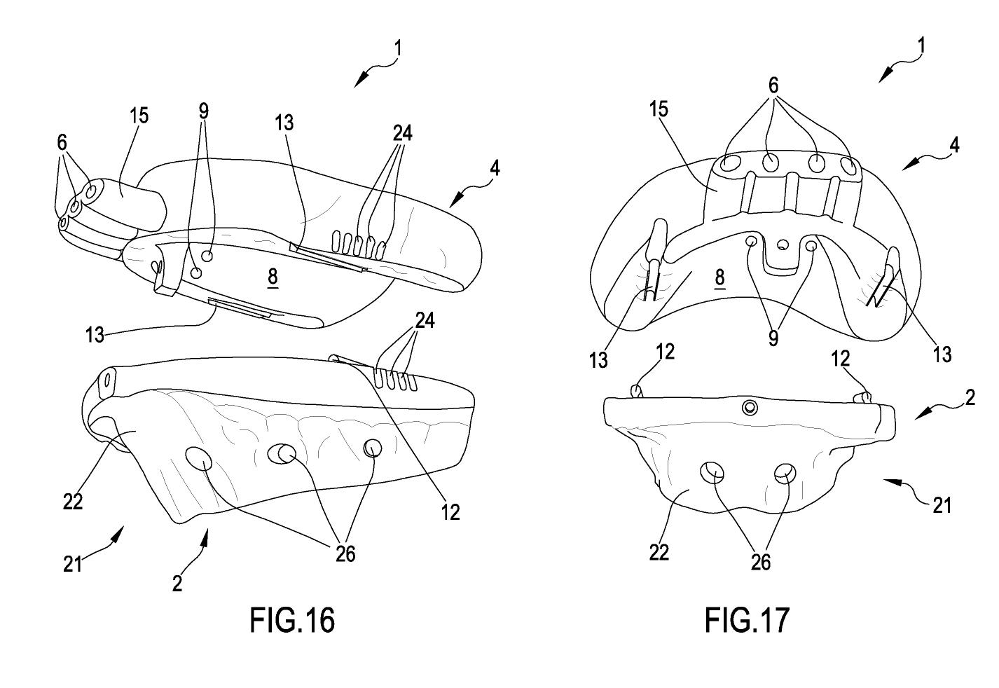

FIGS. 16 and 17 represent front and side perspective views, respectively, of a bite guard according to a sixth embodiment, with the lower and upper portions spaced apart;

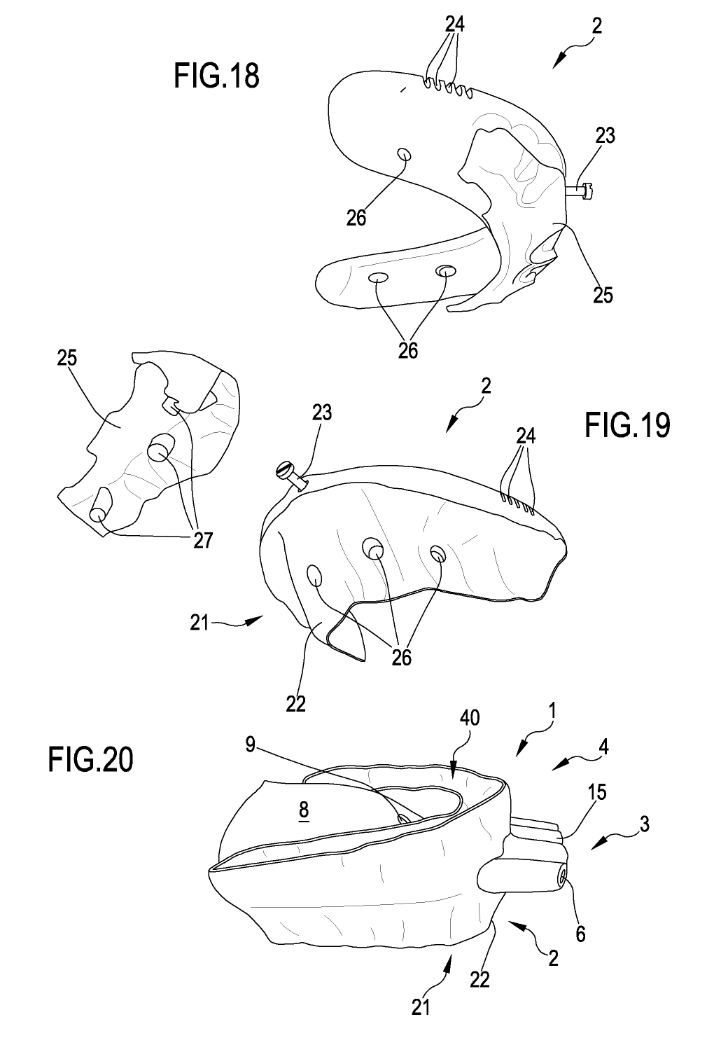

FIG. 18 represents a side perspective view from below of the lower portion of the bite guard according to the sixth embodiment, to which a deformable portion is assembled;

FIG. 19 represents an exploded side perspective view illustrating the deformable portion and the lower portion of the bite guard according to the sixth embodiment;

FIG. 20 represents a side perspective view of a bite guard according to a seventh embodiment;

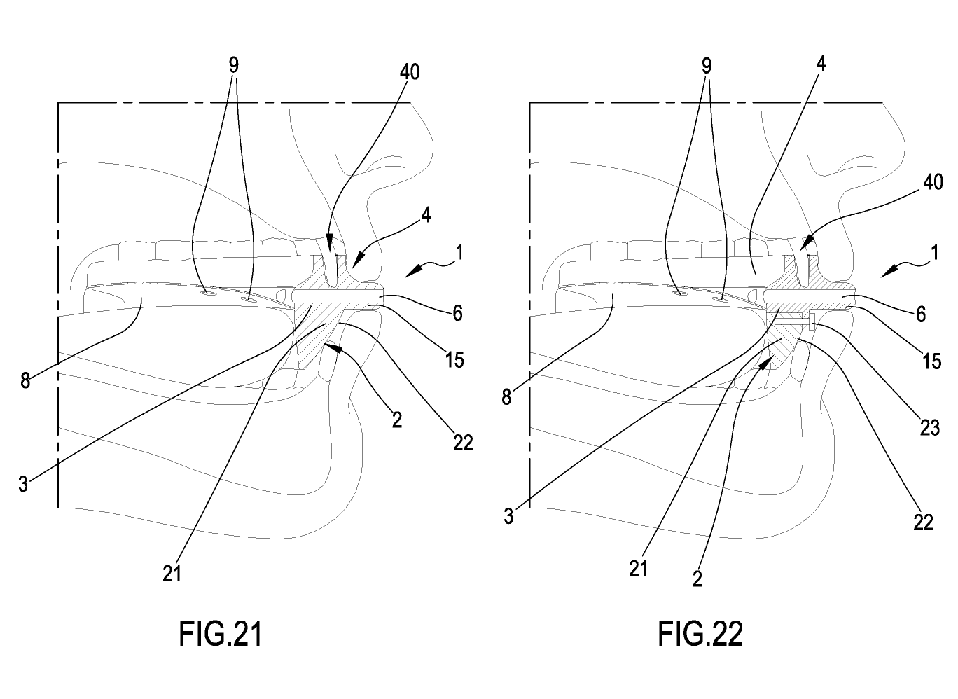

FIG. 21 represents a bite guard according to the seventh embodiment under exemplary conditions of use; and

FIG. 22 represents a bite guard according to the sixth embodiment under exemplary conditions of use.

With reference to the figures, 1 denotes in its entirety a bite guard according to the present disclosure. Under conditions of use, the bite guard 1 is intended to be engaged inside the oral cavity of a wearer or patient in such a way as to bring about a correct relative position between the upper dental arch and the lower dental arch, as will be described in greater detail below.

The bite guard 1 includes a lower portion 2, a central portion 3 and an upper portion 4. The bite guard 1 further includes a positioning element 7, 21 for positioning at least one dental arch of the wearer; in the embodiments from the first to the fifth, the positioning element consists of an abutment ridge 7, whilst in the sixth and seventh embodiments the positioning element consists of a guide portion 21.

The first to fifth embodiments of a bite guard according to the concepts herein will now be described. According to these embodiments, the lower portion 2 is at least partially of a shape mating that of the lower dental arch of the wearer and can comprise a housing seat 20 for the lower dental arch (see FIGS. 9-11). Extending between the lower portion 2 and upper portion 4 is the central portion 3, which can be provided with a plurality of through holes 5 or through channels 6 intended to place, under conditions of use of the bite guard 1, the oral cavity of the wearer in communication with the outside environment to enable the passage of air.

The upper portion 4 includes an abutment ridge 7 where, under conditions of use of the bite guard 1, the front surfaces of the teeth of the upper dental arch are intended to abut. The abutment ridge 7 is vertically aligned with or set rearwardly relative to the lower portion 2; this positioning of the abutment ridge 7 enables the upper arch of the wearer to be positioned, under conditions of use of the bite guard 1, in an aligned position or one that is rearward relative to the lower dental arch. FIG. 11, for example, illustrates a bite guard 1 whose abutment ridge 7 is substantially aligned with the lower portion 2 and, consequently, as illustrated, under conditions of use of the bite guard 1 the upper dental arch of the wearer is substantially aligned with the lower dental arch.

At the time of introducing the bite guard 1 into the oral cavity, the wearer positions the lower portion 2 of the bite guard 1 on the lower dental arch; to enable the positioning of the upper dental arch against the abutment ridge 7, the wearer's lower jaw slides forward relative to the upper jaw. At this point, the wearer positions the teeth of the upper dental arch against the abutment ridge 7 by an appropriate relative protrusive movement between the upper jaw and lower jaw. Under conditions of use of the bite guard 1, at least the incisors of the wearer are resting against the abutment ridge 7. The abutment ridge 7 thus acts as a limit stop for the teeth of the upper dental arch, consequently enabling a correct positioning of the upper dental arch relative to the lower dental arch. Advantageously, in order to enable an ergonomic abutting for the upper dental arch of the wearer, the abutment ridge 7 can be at least partially of a mating shape relative to a plurality of teeth of the upper dental arch.

In multiple embodiments, the abutment ridge 7 can be fixed or movable relative to the lower portion 2.

FIGS. 1-2 illustrate a first embodiment of the bite guard 1. The bite guard 1 according to the first embodiment is made in a single piece of resin and the abutment ridge 7 is fixed relative to the lower portion 2.

FIGS. 3-4 illustrate a second embodiment of the bite guard 1. The bite guard 1 according to the second embodiment includes an arcuate containment portion 8 intended, under conditions of use of the bite guard 1, to contain the wearer's tongue. In other words, under conditions of use of the bite guard 1, the wearer's tongue is positioned beneath the arcuate containment portion 8. This positioning prevents the tongue from moving from the normal lowered position to a raised position, as can generally occur when the wearer is in a supine position (for example, in rest conditions) and thus prevents the tongue from occluding the oropharynx of the wearer. As is well known, such a movement of the tongue can provoke, sleep apnea and roncopathy (or snoring). In light of this, providing a containment portion 8 for the tongue enables sleep apnea and roncopathy to be eliminated or reduced.

The containment portion 8 for the tongue can be provided with through openings 9, which, under conditions of use of the bite guard 1, place a lower portion of the oral cavity of the wearer in communication with an upper portion of the oral cavity, thus permitting, for example, the passage of saliva. As illustrated in FIGS. 3 and 4, the bite guard 1 according to the second embodiment further includes abutment elements 10 laterally disposed on the lower portion 2 and projecting relative thereto.

Under conditions of use of the bite guard 1, the abutment elements 10 are in contact with one or more teeth of the lower dental arch of the wearer so as to bring about a correct positioning relative to the lower portion 2 and limit undesirable movements of the lower dental arch of the wearer. The abutment elements 10 can be made of metal material. Preferably, the abutment elements 10 are at least partially embedded in the lower portion 2. According to the second embodiment, the abutment ridge 7 is fixed relative to the lower portion 2; preferably, the lower portion 2, the central portion 3 and upper portion 4 are made in a single piece of resin.

FIGS. 5-6 and FIGS. 7-8 illustrate a third and a fourth embodiment of the bite guard 1, respectively. The bite guard 1 according to the third and fourth embodiments includes a containment portion 8 for the tongue (as in the second embodiment) and a fixed ridge 11 disposed frontally and externally relative to the abutment ridge 7. According to the third and fourth embodiments, furthermore, the abutment ridge 7 is of a type that is movable relative to the lower portion 2 and relative to the fixed ridge 11. In particular, the abutment ridge 7 can slide rearwardly relative to the fixed ridge 11 in such a way as to operate among a plurality of rearward positions relative to the lower portion 2. The rearward positions are defined at a different distance from the fixed ridge 11 and define, under conditions of use of the bite guard 1, a plurality of possible rearward positions of the upper dental arch of the wearer relative to the lower dental arch. For example, when the abutment ridge 7 is positioned in a nearer or the nearest condition relative to the fixed ridge 11, under conditions of use of the bite guard 1 the teeth of the upper dental arch can be vertically aligned with the teeth of the lower dental arch, whereas as the abutment ridge 7 is progressively moved into a rearward position, the teeth of the upper dental arch take on an increasingly rearward position relative to the teeth of the lower dental arch. Different relative positions of the upper and lower dental arches obviously correspond to different relative positions of the wearer's lower jaw relative to the upper jaw. As the distance between the movable ridge 7 and the fixed ridge 11 increases, the entity of the sliding movement of the wearer's lower jaw relative to the upper jaw increases accordingly. The entity of the protrusive sliding movement of the lower jaw can be determined and set (via the relative distancing between the fixed ridge 11 and movable ridge 7) according to the wearer's specific needs; these needs can be related postural re-education and/or re-education of the positioning of the wearer's lower jaw.

In order to be able to move relative to the lower portion 2, the abutment ridge 7 includes lateral sliding portions 12 at least partially housed in lateral guide portions 13 of the bite guard 1 (see FIG. 12). Upon the movement of the abutment ridge 7, the lateral sliding portions 12 can slide inside the lateral guide portions 13. The sliding of the abutment ridge 7 is controlled by an adjuster 14, which can operate between an adjustment configuration, in which it allows an adjustment of the position of the abutment ridge 7 via the sliding of the lateral portions 13, and a fixed configuration, in which it does not allow an adjustment of the position of the abutment ridge 7. The adjuster 14 can comprise, for example, one or more threaded adjustment elements 140 (see FIGS. 9-10), such as one or more endless screws, configured to control the position of the abutment ridge 7. In other words, the actuation of the adjuster 14 acts on the threaded adjustment elements 140, which act on the abutment ridge 7 so as to bring about the movement thereof. By controlling the positioning of the abutment ridge 7, the adjuster 14 enables the bite guard 1 to be adapted to the wearer's specific needs. FIGS. 9 and 10 illustrate two different operative positions of the abutment ridge; in particular, FIG. 9 illustrates a nearer position of the abutment ridge 7 relative to the fixed ridge 11, whereas FIG. 10 illustrates a more rearward position of the abutment ridge 7 relative to the fixed ridge 11. Starting from the position of the abutment ridge 7 in FIG. 9, the position in FIG. 10 can be reached by acting, precisely, on the threaded adjustment elements 140.

The bite guard 1 according to the third and fourth embodiments further includes an appendage 15 disposed in a front portion of the bite guard 1 and provided with a plurality of through channels 6. The through channels 6 are intended to place, under conditions of use of the bite guard 1, the oral cavity of the wearer in communication with the outside environment to enable the passage of air. Under conditions of use of the bite guard 1, the appendage 15 is disposed between the wearer's lips and provides a rest for the lips themselves (see FIGS. 9 and 10).

In the fourth embodiment, the abutment ridge 7 is made of metal material, whereas in the third embodiment the abutment ridge 7 includes a resin-coated metal skeleton 16. According to the third and fourth embodiments, the upper portion 4, the central portion 3 including the appendage 15, the lower portion 2 and the containment portion 8 for the tongue can be made in a single piece of resin.

According to the third embodiment, as illustrated in FIGS. 5 and 6, the bite guard 1 can further comprise abutment elements 10 (substantially like those of the type envisaged in the second embodiment) configured to bring about the correct positioning of the lower portion 2 relative to the lower dental arch.

FIGS. 13 and 14 illustrate a bite guard 1 according to a fifth embodiment. The bite guard 1 according to the fifth embodiment includes the containment portion 8 for the tongue and the abutment ridge 7 movable relative to the lower portion 2. Unlike the third and fourth embodiments, the bite guard 1 according to the fifth embodiment does not envisage a fixed ridge 11. The abutment ridge 7 is solidly joined to the lateral sliding portions 12, which can slide inside the lateral guide portions 13. The position of the abutment ridge 7 is controlled by actuating a fastening element 17. The fastening element 17 includes a threaded fastening pin 18, which is configured to act on the lateral sliding portions 12 and can be actuated to inhibit the sliding thereof (thus blocking the movement of the abutment ridge 7) or permit the sliding thereof (allowing the movement of the abutment ridge 7).

In a variant embodiment, there can be further provided a metal framework or splint, for example consisting of an appropriately shaped metal wire, engaged with the lower portion and serving to define a plurality of areas of anchorage to the lower dental arch of a wearer, so as to increase the stability and precision of the position of engagement of the bite guard with the lower arch of the wearer.

A sixth and a seventh embodiment of the bite guard 1 will now be described. According to these embodiments, the upper portion 4 is at least partially of a shape mating that of the upper dental arch of the wearer and can comprise a housing seat 40 for the upper dental arch (see FIGS. 20-22). The housing seat 40 serves to enable a stable engagement of the teeth of the upper dental arch with the bite guard 1, in such a way as not to allow, under operative conditions, an undesirable movement of the bite guard 1 inside the oral cavity. The lower portion includes a guide portion 21, which enables a specific relative positioning between the wearer's lower jaw and upper jaw, which will be described in greater detail below. The guide portion 21 extends transversely relative to a front portion of the bite guard 1 and has an inclined surface 22 which extends away from the front portion of the bite guard 1. The extent of the guide portion 21 is clearly illustrated in FIG. 19. The guide portion 21 is preferably solidly joined to the lower portion 2. Upon the introduction of the bite guard 1 into the oral cavity, the wearer positions the upper portion 4 of the bite guard 1 on the upper dental arch; in order to position the lower dental arch, the guide portion 21 guides the lower dental arch, making it slide in the direction of the front portion of the bite guard 1. In this manner, the guide portion 21 enables the wearer's lower jaw to slide forward relative to the upper jaw. At this point, i.e. at the end of the advancement of the lower jaw, the teeth of the lower dental arch of the wearer are positioned frontally relative to the guide portion 21 and the back surfaces of one or more teeth of the lower dental arch are at least partially in contact with the guide portion 21. The bite guard 1 thus permits an appropriate relative protrusive movement between the upper jaw and lower jaw.

According to the sixth embodiment, the lower portion 2 is movable relative to the upper portion 4. By moving the upper portion 4 and the lower portion 2 relative to each other, one modulates the position of the guide portion 21 relative to the upper portion 4. Furthermore, the entity of the sliding protrusive movement of the lower jaw can be determined and set (by the relative distancing between the guide portion 21 and upper portion 4) according to the wearer's specific needs; these needs may be related to postural re-education and/or re-education of the positioning of the wearer's lower jaw.

In order to make the reciprocal movement between the upper portion 4 and lower portion 2 possible, the upper portion 4 is provided with lateral guide portions 13, which act like tracks in which lateral sliding portions 12 of the lower portion 2 can slide. According to one variant (not illustrated in the figures), the lateral guide portions 13 can be disposed on the lower portion 2 and the lateral sliding portions 12 can be disposed on the upper portion 4. The lateral guide portions 13 and the lateral sliding portions 12 can consist of inserts made of metal material integrated into the material (preferably plastic) that the lower portion 2 and upper portion 4 are made of. According to one variant, the lateral guide portions 13 and the lateral sliding portions 12 can be shaped portions made of the same material (preferably plastic) as the lower portion 2 and upper portion 4. The bite guard 1 can further comprise a regulator 23 configured to permit the adjustment of the reciprocal position between the guide portion 21 and upper portion 4. The regulator 23 can be of the threaded type and can comprise a screw, for example an endless screw, for adjusting the position of the guide portion 21 (see FIGS. 19 and 22). As illustrated in FIG. 22, the regulator 23 can be disposed in the central portion 3 of the bite guard 1 and can be accessible (to make an adjustment) from a front portion of the bite guard 1. The regulator 23 is further configured to enable the guide portion 21 to be locked in a specific position.

The bite guard 1 can further comprise position indicators 24 configured to indicate the reciprocal position between the lower portion 2 and upper portion 4. The position indicators 24 are disposed on both the upper portion 4 and lower portion 2 of the bite guard 1 in lateral portions of the bite guard itself (see FIG. 16). In practical terms, even by simply viewing the reciprocal position of the position indicators 24 of the lower portion 2 and upper portion 4, it is possible to determine the position of the guide portion 21 relative to the upper portion 4. According to the seventh embodiment, the lower portion 2 is fixed relative to the upper portion 4. In other words, in the seventh embodiment the guide portion 21, which is solidly joined to the lower portion 2, cannot move relative to the upper portion 4. For example, the bite guard 1 can be made in a single piece, preferably from plastic material. The sixth and the seventh embodiments can share additional features, which are described below. The guide portion 21 of the bite guard 1 can extend for a width that is substantially equal to the extent of the lower dental arch of the wearer, in such a way as to simplify the forward sliding of the lower jaw and enable, under operative conditions of the bite guard 1, an optimal resting of a plurality of teeth of the lower dental arch. According to a variant not illustrated in the appended figures, the guide portion 21 can extend solely for a limited width, substantially equal, for example, to a central portion 3 of the lower dental arch of the wearer corresponding at least to the position of the incisors and canines.

The bite guard 1 can have a deformable or soft portion 25, which can have a substantially gelatinous consistency. The deformable portion 25 has less hardness than the lower portion 4 and is applied frontally relative to the lower portion 4. The deformable portion 25 provides a comfortable rest for the back surfaces of the teeth of the lower dental arch of the wearer and can deform so as to take on a profile corresponding to the conformation of the teeth themselves.

The deformable portion 25 is engaged with the lower portion 2 at one or more through holes 26 of the guide portion 21, extending inside which are precisely the protuberances 27 of the deformable portion 25, which keep it engaged with the guide portion 21. Alternatively, the deformable portion 25 can be engaged with the guide portion 21 by adhesive substances or other mechanical solutions.

The deformable portion 25 can have an extent in width that is substantially equal to that of the lower portion or have a smaller width (see FIG. 18).

The bite guard 1 according to the sixth and seventh embodiments can further have a containment portion 8 for the wearer's tongue of the previously described type, which can have a plurality of through openings 9.

In addition, the bite guard 1 can have an appendage 15, of the previously described type, disposed in a front portion of the bite guard 1 and provided with a plurality of through channels 6 (see FIGS. 16, 17 and 20-22).

The concepts disclosed herein make it possible to obtain one or more of the following advantages and to solve one or more of the problems observed in the prior art.

The concepts disclosed herein provide a bite guard capable of correctly re-educating the position of the wearer's lower jaw and upper jaw.

The concepts disclosed herein provide a bite guard that enables the muscle tensions deriving from incorrect positions of the dental arches of the wearer to be decreased.

The concepts disclosed herein provide a bite guard capable of re-educating the wearer's posture, enabling a correct positioning of the dental arches.

The concepts disclosed herein further provide a bite guard that is of simple, fast and inexpensive construction.

1. A bite guard comprising:
an upper portion intended to be engaged, under conditions of use of the bite guard, with the upper dental arch of a wearer; and
a lower portion intended to be positioned, under at least one condition of use of the bite guard, on the lower dental arch of the wearer, the lower portion comprising a guide portion configured and intended to bring about a predetermined controlled forward sliding of the wearer's lower jaw relative to the wearer's upper jaw by sliding the lower dental arch forward on the guide portion and provide at least a partial rest for the back surfaces of a plurality of teeth of the lower dental arch of the wearer.

2. The bite guard according to claim 1, wherein the guide portion has a surface that is inclined relative to a front portion of the bite guard, the inclined surface extending away from the front portion of the bite guard.

3. The bite guard according to claim 1, wherein the guide portion extends for a width that is substantially equal to an extent of the lower dental arch of the wearer.

4. The bite guard according to claim 1, wherein the conformation of the lower portion is fixed relative to the upper portion.

5. The bite guard according to claim 1, wherein the lower portion is movable relative to the upper portion in order to vary the reciprocal position between the lower portion and upper portion and enable a plurality of operative positions of the bite guard, the guide portion being solidly joined to the lower portion, a movement of the lower portion bringing about the movement of the guide portion in such a way as to modulate the position of the guide portion relative to the upper portion and bring about, according to the position taken on by the guide portion, a specific sliding of the lower jaw relative to the wearer's upper jaw.

6. The bite guard according to claim 5, further comprising regulator for adjusting the position of the lower portion, said regulator being configured to enable a variation in the reciprocal position between the lower portion and upper portion and/or wherein the regulator is of a threaded type and includes a screw for adjusting the reciprocal position between the lower portion and upper portion.

7. The bite guard according to claim 5, further comprising one or more position indicators configured to indicate the reciprocal position between the lower portion and the upper portion.

8. The bite guard according to claim 1, further comprising:
lateral guide portions configured to enable the reciprocal movement between the lower portion and upper portion, the lateral guide portions being disposed on either the lower portion or the upper portion,
lateral sliding portions housed inside the lateral guide portions in such a way as to be able to slide relative to the lateral guide portions, the lateral guide portions being disposed on either the lower portion or the upper portion, the sliding of the lateral portions bringing about the relative sliding between the upper portion and guide portion.

9. The bite guard according to claim 8, wherein the lateral guide portions and the lateral sliding portions are made of metal material integrated into the material, preferably plastic material, that the lower portion and upper portion of the bite guard are made of, or wherein the lateral guide portions and the lateral sliding portions are shaped portions made of same material, preferably plastic, as the lower portion and upper portion of the bite guard.

10. The bite guard according to claim 1, further comprising a deformable or soft portion, having less hardness than the lower portion, said deformable portion being applied frontally relative to the lower portion so as to make resting the teeth of the lower arch on the lower portion more comfortable and/or wherein the deformable or soft portion is engaged with the lower portion at one or more through holes of the guide portion.

11. The bite guard of claim 4, wherein the bite guard is made as a single piece.

12. The bite guard of claim 7, wherein the position indicators are disposed at lateral portions of the bite guard.

13. A method of use of a bite guard comprising:
engaging an upper portion of the bite guard with the upper dental arch of a wearer; and
positioning a lower portion of the bite guard on the lower dental arch of the wearer, so that a guide portion of the lower portion brings about a predetermined controlled forward sliding of the wearer's lower jaw relative to the wearer's upper jaw by sliding the lower dental arch forward on the guide portion and provide at least a partial rest for the back surfaces of a plurality of teeth of the lower dental arch of the wearer;
wherein the bite guard comprises:
an upper portion intended to be engaged, under conditions of use of the bite guard, with the upper dental arch of a wearer; and
a lower portion intended to be positioned, under at least one condition of use of the bite guard, on the lower dental arch of the wearer, the lower portion comprising a guide portion configured and intended to bring about a predetermined controlled forward sliding of the wearer's lower jaw relative to the wearer's upper jaw by sliding the lower dental arch forward on the guide portion and provide at least a partial rest for the back surfaces of a plurality of teeth of the lower dental arch of the wearer.

14. The method of claim 13, wherein positioning the lower portion of the bite guard is selected to correct incorrect positions of the lower jaw of a wearer of the bite guard.

15. The method of claim 13, wherein positioning the lower portion of the bite guard is selected to reduce muscle tensions deriving from incorrect positions of dental arches of a wearer of the bite guard.

16. The method of claim 13, wherein positioning the lower portion of the bite guard is selected for postural re-education of a wearer of the bite guard.

17. A method for forming a bite guard, comprising:
providing an upper portion intended to be engaged with the upper dental arch of a wearer; and
providing a lower portion positionable on the lower dental arch of the wearer;
wherein the lower portion comprises a guide portion configured to bring about a predetermined controlled forward sliding of the wearer's lower jaw relative to the wearer's upper jaw by sliding the lower dental arch forward on the guide portion and provide at least a partial rest for the back surfaces of a plurality of teeth of the lower dental arch of the wearer.

18. The method of claim 17, further comprising fixing the spatial relationship between the lower portion and the upper portion.

19. The method of claim 17, wherein the lower portion is movable relative to the upper portion.

20. The method of claim 17, further comprising providing a regulator, and configuring the regulator to enable a variation in the reciprocal position between the lower portion and upper portion of the bite guard.
